(19) United States
(12) Patent Application Publication
(10) Pub. No.: US 2022/0183549 A1
(43) Pub. Date: Jun. 16, 2022

(54) MAGNETIC TRIAL LENS ADAPTERS FOR AR/VR HEADSET

(71) Applicants: Aaron Smith, Brooklyn, NY (US); Carl Block, Solana Beach, CA (US); Mark Harooni, Great Neck, NY (US)

(72) Inventors: Aaron Smith, Brooklyn, NY (US); Carl Block, Solana Beach, CA (US); Mark Harooni, Great Neck, NY (US)

(73) Assignee: Virtual Field, Inc., Brooklyn, NY (US)

(21) Appl. No.: 17/643,721

(22) Filed: Dec. 10, 2021

Related U.S. Application Data
(60) Provisional application No. 63/126,381, filed on Dec. 16, 2020.

(51) Int. Cl. A61B 3/04

(52) U.S. Cl. CPC A61B 3/04

(57) ABSTRACT

The invention provides a method, system, and device for connecting loose trial lenses into a virtual reality (VR) headset, augmented reality (AR) headset, or other head mounted display. The system consists of two components: First, a component that is installed mechanically into the headset and which remains in the headset throughout the duration of its use. Second, a component which holds loose trial lenses and can be inserted and removed from the headset at will. The two components may connect to each other mechanically, magnetically, electrically, or by other means. In the magnetic system, both components contain embedded magnets that allow the second component to hold loose trial lenses inside the headset magnetically.

### REFERENCE TO PRIOR APPLICATIONS

This application claims priority to U.S. Provisional Application No. 63/126,381 filed on Dec. 16, 2020, titled “System and Method for Attaching Ophthalmic Trial Lenses to a Virtual Reality Headset” which is hereby incorporated by reference herein.

### BACKGROUND OF THE INVENTION

### 1. Technical Field

The invention generally relates to a method, system, and device for connecting loose trial lenses into a virtual reality (VR) headset, augmented reality (AR) headset, or other head mounted display. Implementations of the current subject matter can include, but are not limited to, methods consistent with the descriptions provided herein.

### 2. Background Art

Some eye examinations require that loose trial lenses be used to correct for the patient's refractive error. Thus the results of a visual field examination, pupillometry, or other exam are not impacted by the patient's refractive error, and effects of disease, if any exists, are readily apparent.

As the eye care industry adopts virtual reality (VR) head mounted displays as a means of detecting and following the progression of eye disease, a need has arisen for refractive error correction in virtual reality headsets. In some cases, the patient's glasses may be worn inside the headset during the exam. This has the benefit of convenience, but does not allow for accurate vision correction in all cases. In other cases, a custom lens may be purchased which contains the patient's prescription and can be clipped into the headset. This has the benefit of specificity, but does not allow for the flexibility of use for other patients.

In a standard eye exam, loose trial lenses are used to correct for the patient's refractive error. Most eye care providers own a set of loose trial lenses that they can use to correct the patient's vision during an eye exam. They may, for example, use these when prescribing the patient a set of new glasses. The trial lenses would be put in front of the patient's eye, one at a time, until the patient's vision improves. The provider would then write that into the patient's prescription.

Similarly, some medical equipment, for example visual field machines, contain slots for loose trial lenses. These slots are built into the machine, and simply hold one or two trial lenses for the patient to look through during the exam.

In virtual reality (VR) headsets, it is inconvenient and/or impossible to have trial lens slots permanently built into the headset. If the trial lens holders are permanently built into the headset, then the possibility of wearing glasses inside the headset is forgone—there is not enough room for both the glasses and the trial lens holders. Even if glasses are not worn, the trial lens holders take up considerable space inside the headset and may cause either discomfort to the patient's face or nose, or block the patient's view. In other words, the trial lens holders should only be in the headset when strictly necessary to hold trial lenses.

Therefore there exists a need for a method and device that allows for trial lens holders to be inserted and removed from the virtual reality (VR) or augmented reality (AR) headset quickly and easily in a medical setting. Moreover, there is a need for this system to have magnetic or mechanical components that secure the lens holders into place while they are inside the headset, while still being easy to remove when necessary.

### SUMMARY OF THE INVENTION

The invention provides a method, system, and device for connecting loose trial lenses into a virtual reality (VR) headset, augmented reality (AR) headset, or other head mounted display. In particular, there is a base component installed into the headset which may connect to the headset with some mechanical component. There is a removable component that holds the loose trial lenses which may be in the headset or removed. The loose trial lens holder may hold one or more trial lenses. The loose tense holder may attach to the installed base component with magnets. Further, the loose tense holder may attach to the installed base component mechanically.

A first aspect of the invention provides a method for attaching a permanent base component which stays in the headset. This base component may have magnetic or mechanical mechanisms that the removable component can use to attach to it. The base component may connect to the headset itself with mechanical clips, glue, or many other methods.

A second aspect of the invention provides a removable component that holds the trial lenses. The removable component may have magnetic or mechanical features which allow it to attach to the base component and thus be securely attached to the headset.

A third aspect of the invention provides mechanisms for the removable component to bold and secure loose trial lenses. These may be clamps, slots, or other mechanisms.

A fourth aspect of the invention provides a method for securing the base and removable components of the invention together with magnets. Both the base and removable components may contain magnets, and these magnets may be lined up in such a way that they can securely attach to each other when the removable component is inserted into the headset. The magnets may be contained inside the components so that they are not visible to the user.

A fifth aspect of the invention provides a method for securing the base and removable components of the invention together with a mechanical system. The mechanical system may contain a screw, clip, plug, or other method for securely attaching the removable component to the base component and thus securely inserting the removable component holding the trial lenses into the headset.

The details of one or more variations of the subject matter described herein are set forth in the accompanying drawings and the description below. Other features and advantages of the subject matter described herein will he apparent from the description and drawings, and from the claims. While certain features of the currently disclosed subject matter are described for illustrative purposes in relation to the hardware, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts trial lenses, which are used by ophthalmologists, optometrists, and others to correct for refractive error and are included here only for descriptive purposes;

FIG. 2 depicts a complete mechanical system for attaching these trial lenses into a virtual reality (VR) headset;

FIG. 3 depicts a component that attaches to the virtual reality (VR) system and has extruded magnetic surfaces where another component can attach;

FIG. 4 illustrates one method of attaching the component to the virtual reality (VR) system by using small protrusions that connect to the virtual reality (VR) device;

FIG. 5 depicts a removable component that has two magnetic surfaces that can connect to the component that is permanently attached to the virtual reality (VR) device;

FIG. 6 depicts slots for loose trial lenses to be inserted and secured;

FIG. 7 illustrates how magnets can be embedded into the base component as one method of securing the removable component;

FIG. 8 illustrates one method of connecting the permanent and removable components using magnets;

FIG. 9 illustrates how magnets can be embedded into the removable component as one method of securing the removable component;

FIG. 10 illustrates how a trial lens adapter can be connected inside a virtual reality (VR) headset.

### DETAILED DESCRIPTION

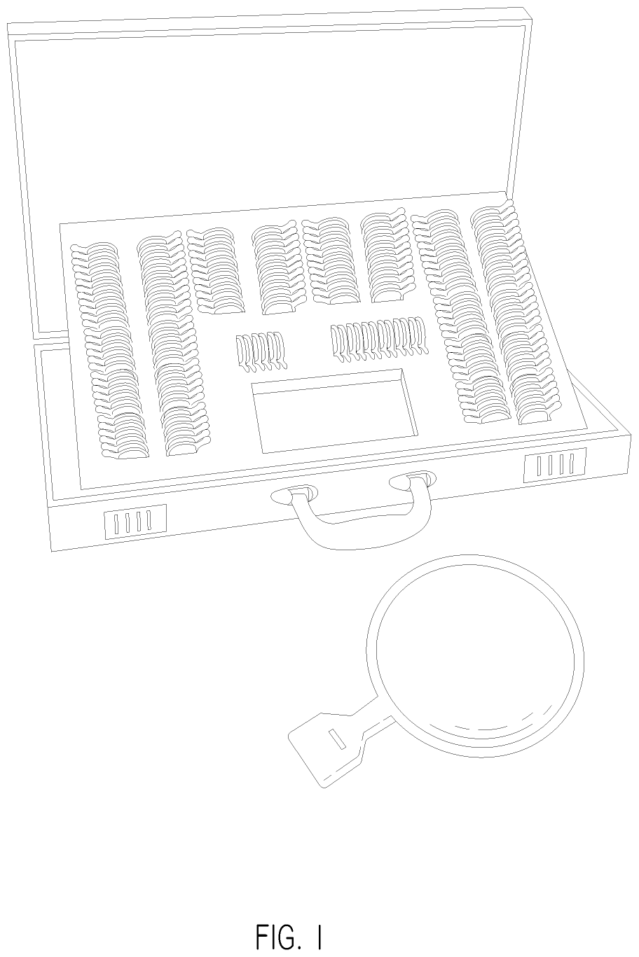

FIG. 1 is included for descriptive purposes only and depicts loose trial lenses, which are used by eye care professionals to find the best prescription for a patient or correct for the patient's refractive error during a diagnostic exam.

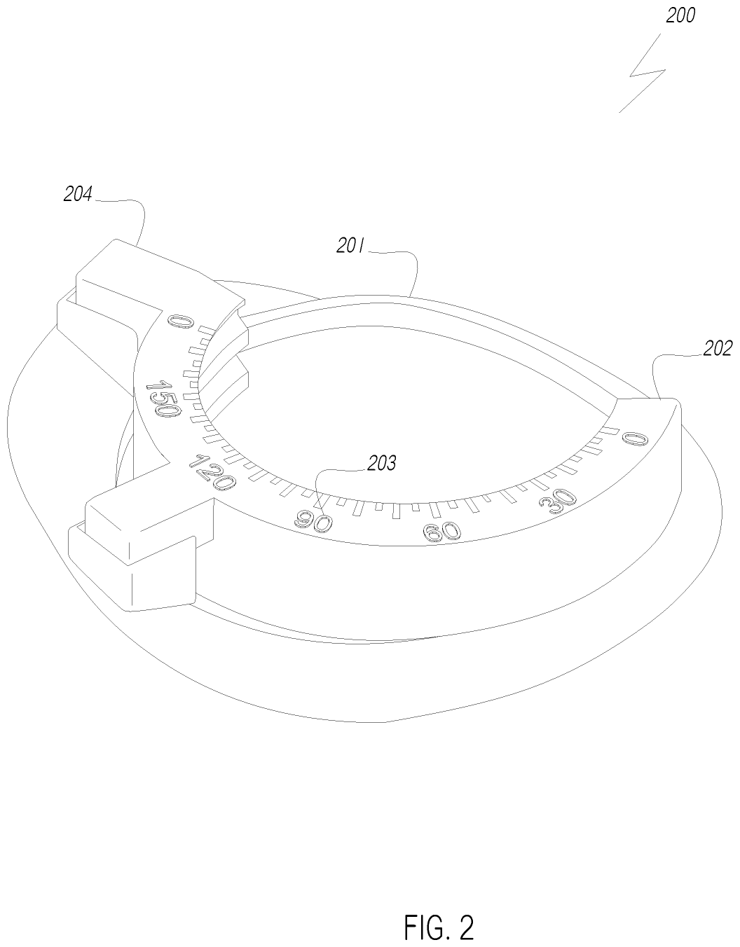

FIG. 2 depicts a lens adapter system 200, in accordance with some example embodiments. The system 200 can include a base component 201 and a detachable component 202, among other features. The detachable component 202 may include a dash and/or numbering system 203 which demarcates the degrees at which the trial lenses sit. As shown in FIG. 2, the base component 201 and the detachable component 202 may be attached via a mechanical mechanism. In this example, magnets are used to attach the two components together, but other mechanical systems may be devised.

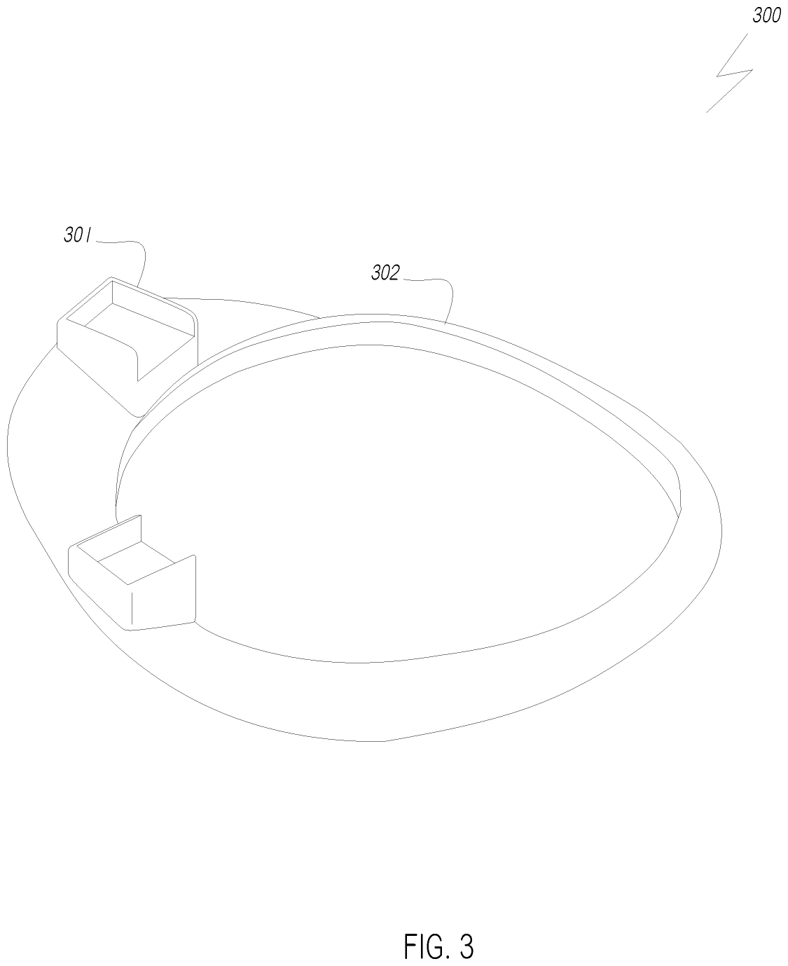

FIG. 3 depicts lens adapter base component 300, in accordance with some example embodiments. The base component 300 may include a mechanical attachment point 301 where the detachable component can attach. This attachment point can be mechanical, electrical, magnetic, or otherwise. The base component 300 may also include a virtual reality (VR) headset specific attachment surface 302. This attachment surface may be specific to the virtual reality (VR) headset that the lens adapter component is being attached to.

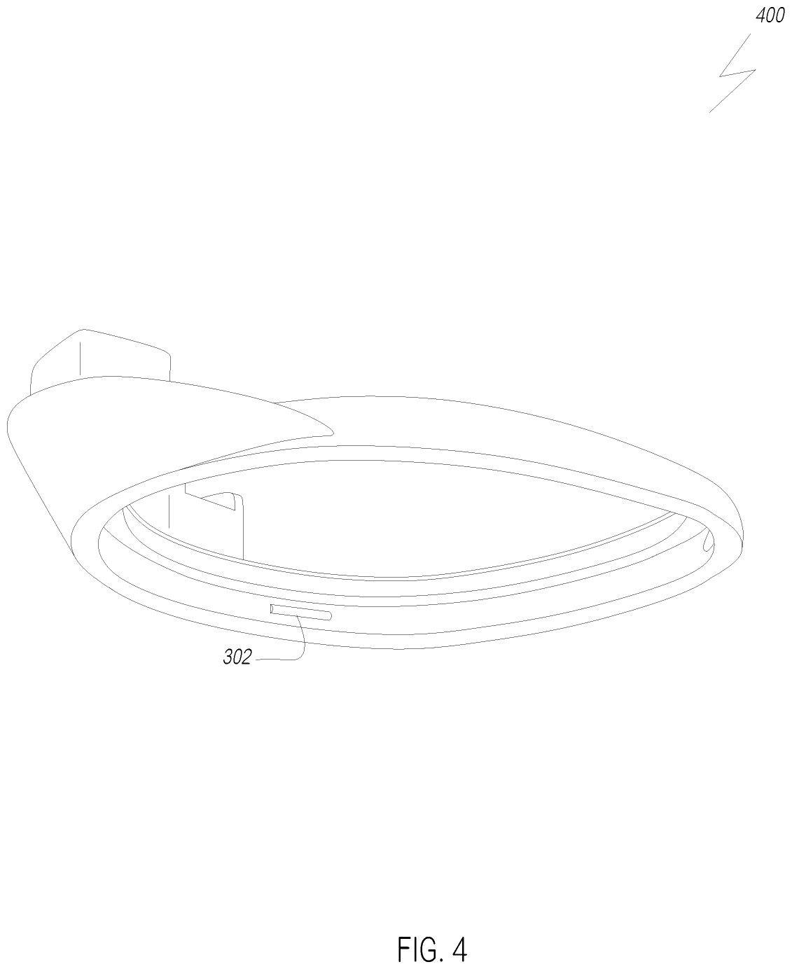

FIG. 4 depicts lens adapter base component 400, in accordance with some example embodiments. The base component 400 may include mechanical attachment point 401 where the base component connects to the virtual reality (VR) headset. Such attachment point(s) may be different for each headset, or may be the same between headsets.

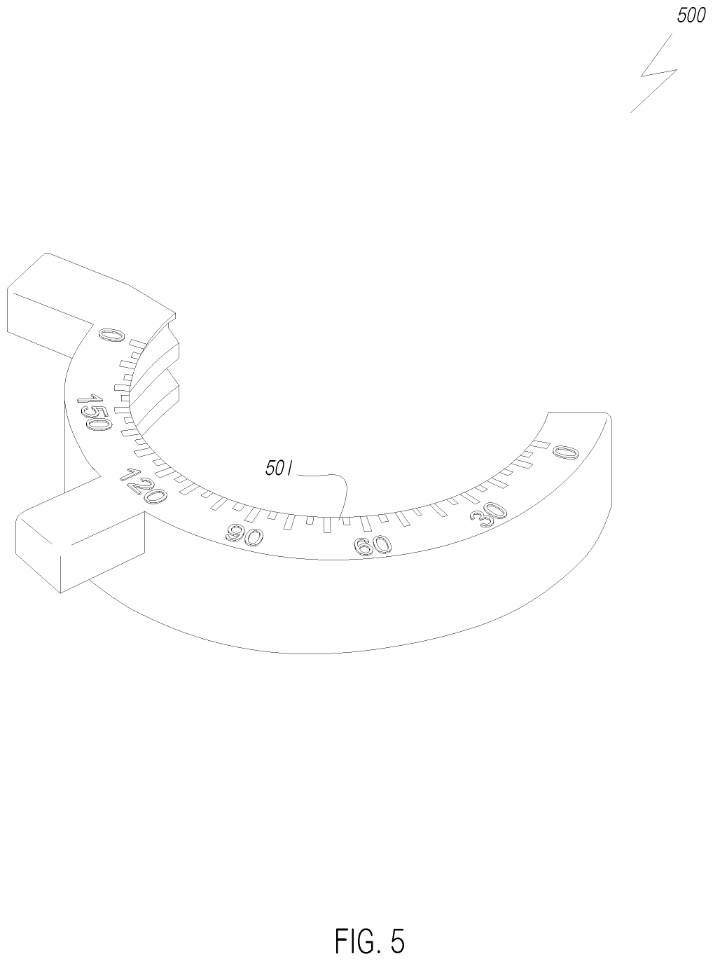

FIG. 5 depicts lens adapter detachable component 500, in accordance with some example embodiments. The detachable component 500 may include a dash and/or numbering system 501 which demarcates the degrees at which the trial lenses sit.

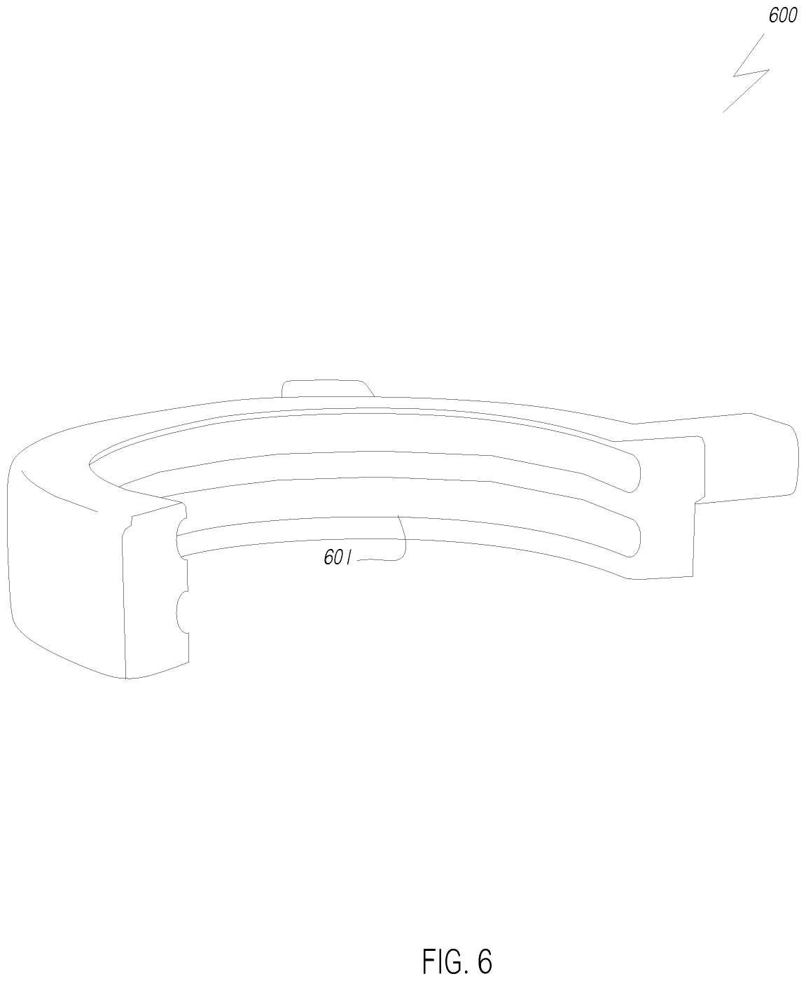

FIG. 6 depicts lens adapter detachable component 600, in accordance with some example embodiments. The detachable component 600 may include one or more trial lens slots 601. These trial lens slots are made to hold standard trial lenses.

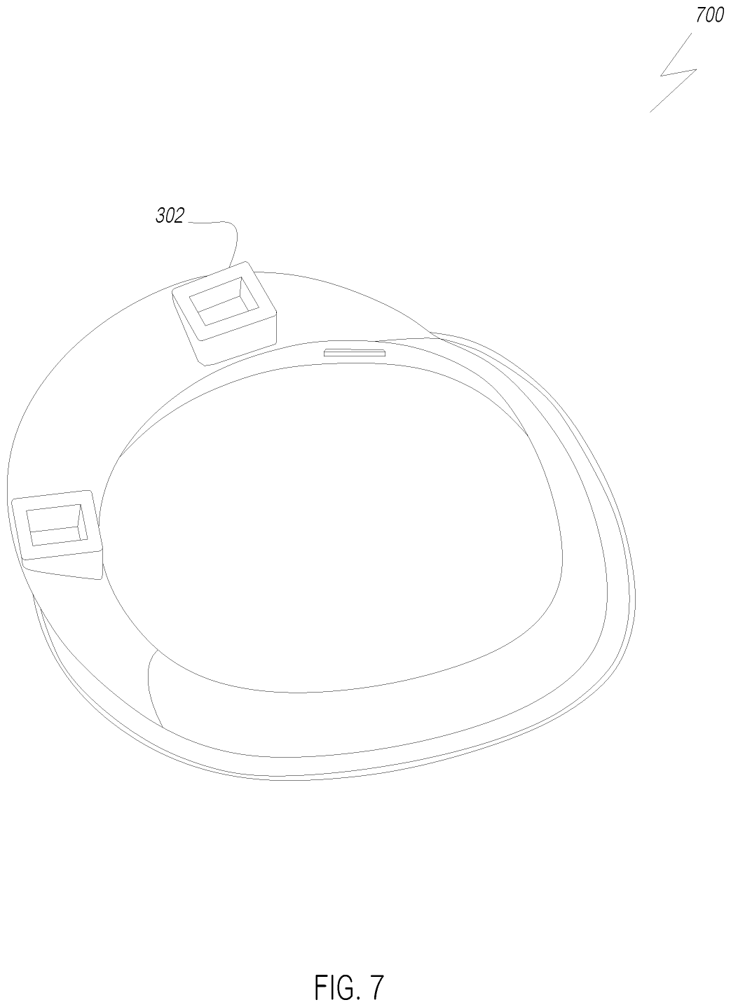

FIG. 7 depicts lens adapter base component 700, in accordance with some example embodiments. The base component 700 may include magnet hole 701, or any number of other mechanical attachment methods, in order to secure it to the base component during an eye exam.

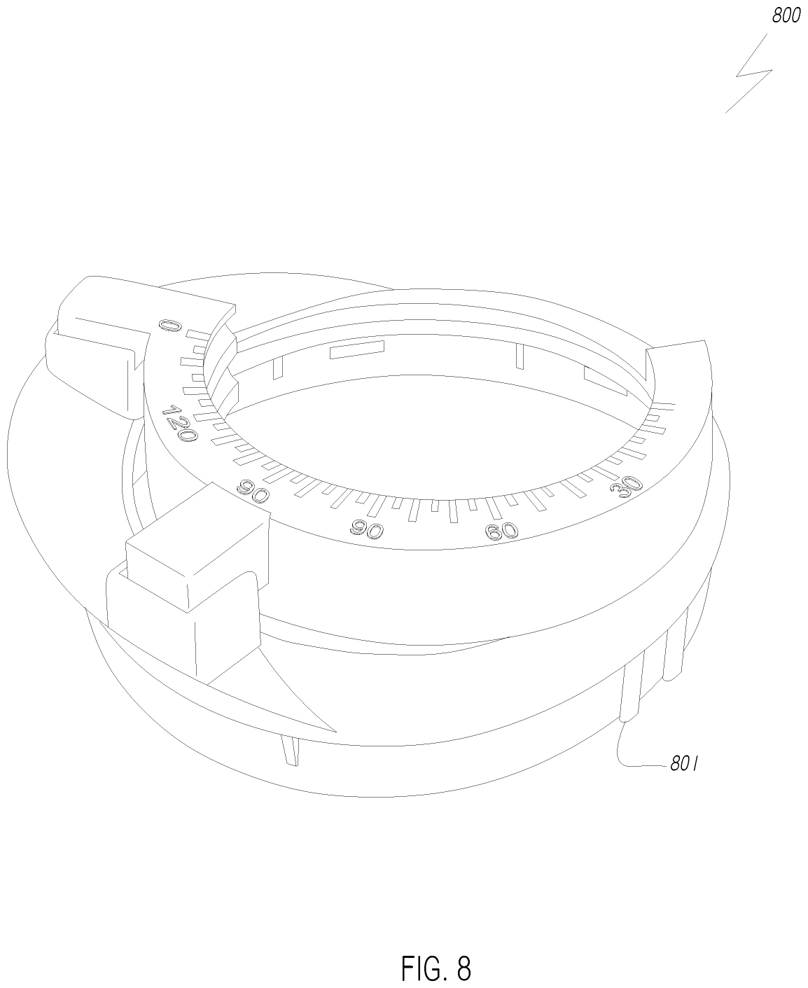

FIG. 8 depicts a lens adapter system 800, in accordance with some example embodiments. The system 800 demonstrates an attachment mechanism 801 for an alternative headset than the one shown previously for demonstrative purposes.

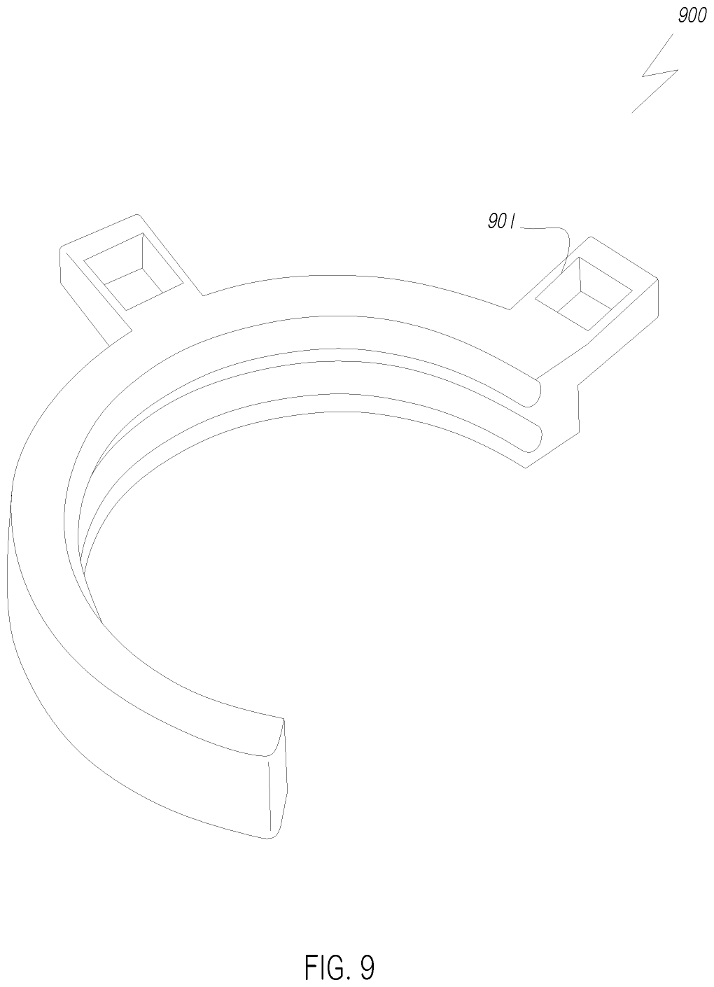

FIG. 9 depicts a lens adapter detachable component 900, in accordance with some example embodiments. The detachable component 900 may include magnet hole 901, or any number of other mechanical attachment methods, in order to secure it to the base component during an eye exam.

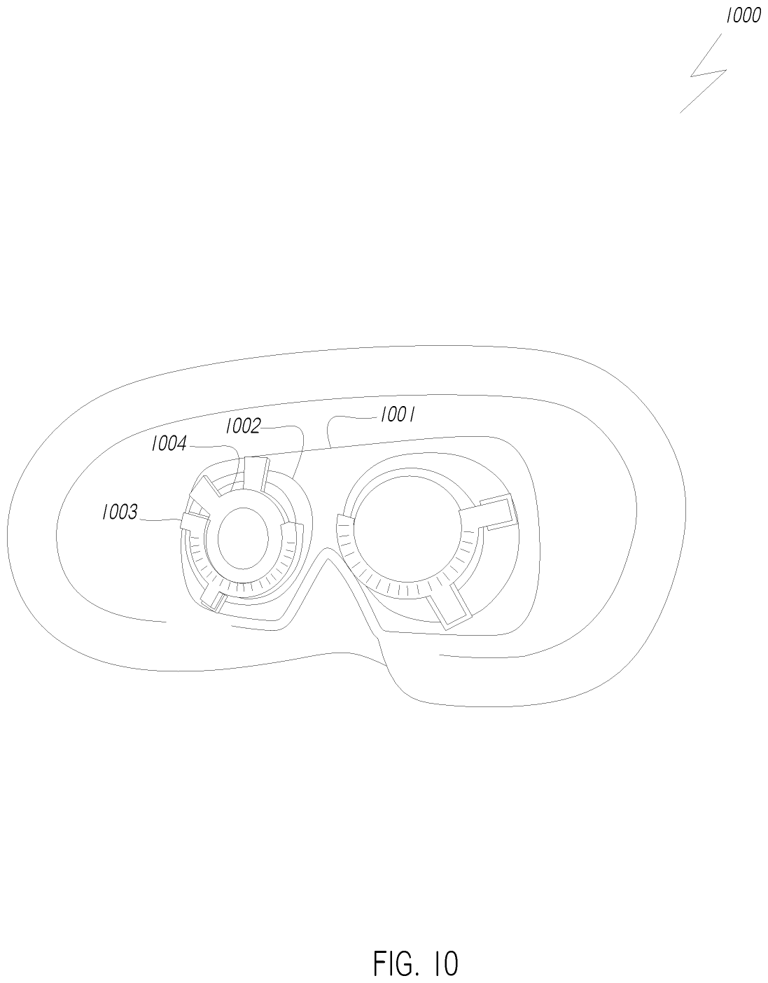

FIG. 10 depicts a lens adapter system 1000, in accordance with some embodiments. Base component 1002 is connected to virtual reality (VR) headset 1001. Lens adapter detachable component 1003 is attached to base component 1002 by magnets in this example. Loose trial lenses 1004 are held in the detachable component 1003.

### Background

FIG. 1 depicts both single and a set of loose trial lenses. Loose trial lenses are used by eye care professionals (optometrists, ophthalmologists, etc.) in order to both diagnose vision problems—for example, prescribing glasses—and for correcting vision problems during an examination where refractive error must be isolated. An example of this is during a visual field test, where the clinician will use trial lenses to isolate the patient's refractive error and measure the eye's sensitivity to light in order to diagnose glaucoma and other diseases.

### Trial Lens Adapters

FIG. 2 depicts a possible design for a detachable system that allows for a clinician to insert trial lenses into a virtual reality (VR) headset for use during an eye exam. There is a base component 201 that is clipped or permanently attached to the virtual reality (VR) headset

The base component 201 may use different interfaces to connect to different headsets. Examples are interfaces 401 and 801. The interface may also be built into the headset permanently.

The base component 201 has one or more mechanical attachment points 301 where the detachable component is connected. The mechanical attachment point may use a physical force, such as friction, in order to keep the detachable component in place, or it may include embedded magnets as is demonstrated in 701, or it may use another mechanical method not specifically outlined here.

FIG. 2 depicts detachable component 202. Detachable component 202 has one or more attachment points 204 where the detachable component connects to the base component. The mechanical attachment point may use a physical force, such as friction, in order to keep the detachable component in place, or it may include embedded magnets as is demonstrated in 901, or it may use another mechanical method not specifically outlined here.

The clinician can remove detachable component 202 and insert trial lenses into the trial lens slots 601 which are in the component There can be one or more slots for lenses. A clinician may insert one lens, or they may insert more than one lens, depending on the patient's vision

The virtual reality (VR) headset can be used with or without the detachable component 202. When detachable component 202 is not in use, the patient may use their own glasses or contacts in the headset. When detachable component 202 is used, the patient can look through loose trial lenses that are held in detachable component 202.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A system comprising:
at least one base component that is installed in a headset by mechanical means;
at least one removable component that can be attached and removed from the base component by magnetic means; and
at least one way to insert and retain loose trial lenses in the removable component by mechanical means.

2. The system of claim 1 wherein the removable component can be attached and removed from the base component by mechanical means.

3. The system of claim 1 wherein the removable component can be attached and removed from the base component by electrical means.

4. The system of claim 1 wherein the base component is installed in the headset by magnetic means.

5. The system of claim 1 wherein the loose trial lenses are inserted and retained by magnetic means.

6. The system of claim 1 wherein the loose trial lenses are inserted and retained by electric means.

7. The system of claim 1 wherein each eye in the headset has a separate set of base and removable components.

8. The system of claim 1 wherein both eyes in the headset have one combined set of base and removable components.

9. A system comprising:
At least one base component that is installed in a headset by mechanical means;
At least one way to insert and retain loose trial lenses in the base component by mechanical means.

10. The system of claim 7 wherein the base component is installed in the headset by magnetic means.

11. The system of claim 7 wherein the base component is installed in the headset by electric means.

12. The system of claim 7 wherein the loose trial lenses are inserted and retained in the base component by magnetic means.

13. The system of claim 7 wherein the loose trial lenses are inserted and retained in the base component by electric means.

14. The system of claim 7 wherein each eye in the headset has a separate base component.

15. The system of claim 7 wherein both eyes in the headset have one combined base component.
